(19) United States
(12) Patent Application Publication
(10) Pub. No.: US 2024/0252796 A1
(43) Pub. Date: Aug. 1, 2024

(54) MEDICAL DEVICE WITH FLUID ACIVATED ACTUATION MECHANISM

(71) Applicant: Novo Nordisk A/S, Bagsvaerd (DK)

(72) Inventor: Noekkvi Steinn Sigurdarson, Koebenhavn N (DK)

(21) Appl. No.: 18/566,555

(22) PCT Filed: Jun. 9, 2022
(86) PCT No.: PCT/EP2022/065628

§ 371 Date: Dec. 1, 2023

Related U.S. Application Data
(60) Provisional application No. 63/212,239, filed on Jun. 18, 2021.

(51) Int. Cl. A61M 31/00

(52) U.S. Cl. CPC A61M 31/002

(57) ABSTRACT

A self-righting ingestible capsule comprising: a) a capsule housing (110, 120) defining a central axis and having proximal and distal ends, b) a sealing compartment (A) within the capsule housing (110, 120) accommodating a solid dose member (130), and comprising a distal exit port (124), c) an actuation compartment (B) fluidically isolated from the scaling compartment (A), d) an actuation member (150) at least partially accommodated in the actuation compartment (B) and arranged for axial displacement thereby moving the dose member (130) distally through the distal exit port (124), e) a helical spring (140) for powering the actuation member (150) in a distal direction, f) a latch component (152) coupled to the actuation member (150) for releasably maintaining the actuation member latched in a proximal position, and g) a dissolvable latch support (160) disposed in the actuation compartment (B), wherein the dissolvable latch support maintains latching of the actuation member (150) in the proximal position by supporting the latch component

The present invention relates to medical devices, including systems for drug delivery, adapted for being inserted into a lumen of a patient and capable of being activated by means of a fluid, such as a biological fluid.

### BACKGROUND OF THE INVENTION

In the disclosure of the present invention reference is mostly made to the treatment of a disease or other condition by delivery of a therapeutic agent, however, this is only an exemplary use of the present invention.

May people suffer from diseases, which requires them to receive injections of drugs on a regular and often daily basis. To treat their disease these people are required to perform different tasks which may be considered complicated and may be experienced as uncomfortable. Furthermore, it requires them to bring injection devices, needles and drugs with them when they leave home. It would therefore be considered a significant improvement of the treatment of such diseases if treatment could be based on oral intake of tablets or capsules.

However, such solutions are very difficult to realise since protein-based drugs will be degraded and digested rather than absorbed when ingested.

To provide a working solution for delivering a therapeutic substance into the bloodstream through oral intake, the drug has to be delivered firstly into a lumen of the gastrointestinal tract and further into the wall of the gastrointestinal tract (lumen wall). This presents several challenges among which are: (1) The drug has to be protected from degradation or digestion by the acid in the stomach. (2) The drug has to be released while being in the stomach, or in the lower gastrointestinal tract, i.e. after the stomach, which limits the window of opportunity for drug release. (3) The drug has to be delivered at the lumen wall to limit the time exposed to the degrading environment of the fluids in the stomach and in the lower gastrointestinal tract. If not released at the wall, the drug may be degraded during its travel from point of release to the wall or may pass through the lower gastrointestinal tract without being absorbed, unless being protected against the decomposing fluids.

Prior art references relating to oral dosing of active agents and addressing one or more of the above challenges include WO 2018/213600 A1, WO 2020/160399 A1 and US 2020/0129441 A1.

For medical capsules, such as the ones disclosed in the said references, the internal configuration design offers several design challenge trade-offs. For an oral device to be viable, e.g. for delivery of an API in form of a solid needle-shaped API, it needs to deliver an amount of API sufficient for the intended therapy. At the same time, for solid needle-shaped API tablets, the API tablet needs to be delivered reliably into a tissue layer in a depth sufficient to enable systemic uptake. Typically, a large injection force is required to deliver the API tablet at the right depth. Hence, the challenge is to design a device that is small enough to be swallowable, while reliably self-righting and injecting a sufficient amount of API deep enough. Furthermore, low cost and robust performance is essential.

In WO 2020/157324 A1 a capsule device is disclosed having an actuation mechanism with a laterally movable latch element which includes a blocking portion that cooperates with a retainer portion in latching engagement. Prior to actuation the latch element is supported by a dissolvable retaining member, which upon dissolution ceases to support the latch element thereby enabling lateral movement or the latch element to release the latching engagement.

For this kind of actuation mechanism, typical constraints and requirements for obtaining a well-functioning mechanism include the following:
the ability to maintain an actuation member against the load of a drive spring for a prolonged time, even for a drive spring exerting a load of large magnitude.
the actuation member shall be kept in place during dissolving of the dissolvable retaining member in order to maintain the desired acceleration when actuated, and
the dissolvable retaining member needs to be totally dissolved to avoid undissolved pieces that could block the actuation mechanism.

Having regard to the above, it is an object of the present invention to provide a medical device for insertion into a lumen of a patient, and which to a high degree effectively and reliably allows firing of an actuation mechanism in a controlled and predictable manner by influence of a biological fluid.

It is furthermore an object of the present invention to provide a capsule design which is optimized for accommodating a large dosage of payload, that provides a forceful actuation force, and wherein the outer dimensions of the capsule are minimized.

### DISCLOSURE OF THE INVENTION

In the disclosure of the present invention, embodiments and aspects will be described which will address one or more of the above objects or which will address objects apparent from the below disclosure as well as from the description of exemplary embodiments.

Thus, in a first aspect, a self-righting ingestible capsule is provided, comprising:
a capsule housing defining a central axis and having proximal and distal ends,
a sealing compartment (A) within the capsule housing, the sealing compartment (A) accommodating a solid dose member and comprising a distal exit port, and
an actuation compartment (B) fluidically isolated from the sealing compartment (A), wherein a self-actuating actuator is at least partially accommodated in the actuation compartment (B),
   wherein the self-actuating actuator comprises:
an actuation member arranged for axial distal displacement thereby moving the dose member distally through the distal exit port,
a helical spring biasing the actuation member in a distal direction,
a latch component coupled to the actuation member for releasably maintaining the actuation member latched in a proximal position against the bias of the helical spring, and a dissolvable latch support disposed in the actuation compartment (B), wherein prior to dissolution, the dissolvable latch support maintains latching of the actuation member in the proximal position by supporting the latch component, and upon dissolution, said supporting of the latch component ceases thereby enabling the actuation member to be displaced distally, wherein, prior to dissolution, an annular seal maintains the sealing compartment (A) fluidically isolated from the actuation compartment (B), the annular seal being arranged radially outwards relative to at least a portion of the helical spring.

In certain embodiments the self-righting capsule defines a capsule suitable for ingestion into a lumen of a patient, the lumen having a lumen wall, the self-righting capsule comprising:
a capsule housing defining a central axis and having proximal and distal ends,
a sealing compartment (A) within the capsule housing for accommodating a dose member shaped as a solid therapeutic dosage form formed partly or entirely from a preparation comprising a therapeutic payload, wherein the sealing compartment (A) comprises an axially extending enclosure having a distal end provided with an exit port and a proximal end, and
an actuation compartment (B) fluidically isolated from the sealing compartment (A) and at least partially accommodating a self-actuating actuator,
   wherein the self-actuating actuator comprises:
an actuation member arranged for axial displacement relative to the axially extending enclosure from a proximal position to a distal position thereby cooperating with the dose member for moving the dose member distally from a non-exposed position within the sealing compartment (A) to an exposed position wherein at least a portion of the dose member is exposed through the exit port,
a helical spring for displacing the actuation member, wherein the helical spring is strained to exert a load on the actuation member for displacement from the proximal position to the distal position,
a latch component coupled to the actuation member for releasably maintaining the actuation member latched in the proximal position, and
a dissolvable latch support disposed in the actuation compartment (B), the dissolvable latch support being at least partially dissolvable when subjected to a biological fluid entering the actuation compartment (B), wherein the dissolvable latch support, prior to dissolution, maintains latching of the actuation member in the proximal position by supporting the latch component, and wherein at least partial dissolution of the dissolvable latch support causes support of the latch component to cease thereby enabling the actuation member to be displaced towards the distal position,
   wherein the self-righting capsule comprises a sealed interface between the actuation member and the axially extending enclosure that, with the actuation member assuming the proximal position, maintains the sealing compartment (A) fluidically isolated from the actuation compartment (B), wherein the sealed interface comprises an annular seal arranged radially outwards relative to at least a portion of the helical spring.

Compared to previously suggested self-righting capsules disclosed in the art, the present solution differ in that the annular seal has been moved radially outside the pre-strained spring. The inventor of the present invention has recognized that the proposed solution offers an improved solution wherein several or even all of the aspects of capsule dimension, drug load, self-righting ability and drug insertion velocity can be further optimized. Besides the load bearing surfaces of the self-actuating actuator can be optimized, generally increasing the performance of the self-righting capsule.

These changes allow the spring force (and thereby the injection depth) to be increased, and the device size to be reduced. All the while improving control over the dissolution of the dissolvable latch support.

The helical spring may be provided in the form of a compression spring which forms a plurality of windings. In some embodiments, a distal portion of the windings defines an outside diameter of first magnitude, wherein the annular seal comprises a radially inwards facing surface defining an inner diameter of second magnitude larger than the first magnitude.

In some embodiments the helical spring is provided as a coiled tapered spring having a wide proximal end coupled to the capsule housing and a narrow distal end coupled to actuation member. This enables a spring which is stiffer, provides more energy for moving the dose member into tissue, and which provides advantages with respect to handling during manufacture.

In other embodiments, the outer shape of the spring may be different, such as cylindrical, i.e. non-tapering.

The annular seal may in some embodiments be formed sealing member, such as a ring-shaped member, and being formed from a sealing material. The sealing material may be formed from soft pliable material, such as an elastomeric material, e.g. an FDA approved rubber, such as silicone containing material.

In some forms the annular seal is arranged in axially overlapping relationship with a portion of the helical spring, at least when the actuation member assumes the proximal position, and/or at least when the actuation member assumes the distal position.

In some forms the annular seal is arranged with a radially outwards facing surface interfacing with a radially inwards surface of the axially extending enclosure.

The annular seal may be arranged so that it is disposed fixedly relative to the axially extending enclosure as the actuation member moves from the proximal position to the distal position. In other embodiments, the annular seal is attached fixedly relative to the actuation member and follows movement of the actuation member.

In some forms the actuation member comprises a distal portion coupled to the dose member and wherein the latch component is attached to or formed integrally with the distal portion, the latch component protruding proximally from the distal portion of the actuation member.

Said latching of the actuation member in the proximal position, i.e. prior to dissolution of the dissolvable latch support, may be provided by engaging support of the latch component by one or more retainer portions associated with the capsule housing. The one or more retainer portions may be arranged such as generally proximally facing surfaces formed either integrally with the capsule housing or being formed by a component fixedly attached to the capsule housing. Typically, the one or more retainer portions are associated with, such as by being formed by, a portion of the self-righting capsule that defines a spring seat for the proximal end of the helical spring. Prior to dissolution, the dissolvable latch support maintains latching engagement between the latch component and the one or more retainer portions.

The latch component may be formed so that it comprises one or more latch arms attached to, or being integrally formed with, the distal portion of the actuation member, the one or more latch arms being formed to extend proximally from the distal portion of the actuation member to cooperate with corresponding retainer portions associated with the capsule housing.

In some embodiments, at least one of said one or more latch arms is formed as a laterally movable latch arm defining a blocking portion that cooperates with a retainer portion in latching engagement, wherein, prior to dissolution of the dissolvable latch support, the latch arm is supported by the dissolvable latch support, which upon dissolution ceases to support the latch arm thereby enabling lateral movement of the latch arm to release the latching engagement.

In further embodiments, the self-righting capsule is formed to comprise a plurality of latch arms wherein each latch arm defines radially outwards and radially inwards facing latch surfaces, and wherein each latch arm is radially deflectable,

wherein the dissolvable latch support is arranged centrally on-axis, wherein the retainer portions are arranged coaxially around the dissolvable latch support, and wherein the latch arms are arranged radially in between,
wherein, prior to dissolution of the dissolvable latch support, the radially outwards facing latch surfaces engage with respective retainer portions in a latching engagement, and the radially inwards facing latch surfaces are in supporting engagement with respective radially outwards facing surfaces of the dissolvable latch support thereby restricting the latch arms from moving radially inwards and preventing release of the latching engagement,
wherein, prior to dissolution, the latch arms extend proximally and radially outwards from the distal portion of the actuation member so that the radially inwards facing latch surface of each latch arm is inclined relative to the axis, and wherein the respective radially outwards facing surfaces of the dissolvable latch support are inclined thereby supporting the respective latch arm along the radially inwards facing latch surface, and
wherein, when the dissolvable latch support has become at least partially dissolved, the latch arms are allowed to move radially inwards thereby releasing the latching engagement and allowing the helical spring to move the actuation member towards the distal position.

The self-righting capsule may define a pre-actuation configuration which corresponds to the state prior to dissolution of the dissolvable latch support. Also, an actuating configuration may define a state where the dissolvable latch support has become at least partially dissolved for release of the actuation member to occur.

By configuring the latch mechanism with latch arms that extend proximally and radially outwards from the base portion so that the radially inwards facing latch surface of each latch arm is inclined relative to the axis this enables, in the actuating configuration, the latch arms to collapse radially toward each other in a manner where the latch arms only takes up little space in the radial direction. This enables the retainer portions associated with the capsule housing to be formed with a radially minor dimension. This in turn enables use of a more powerful drive spring for obtaining a large actuation force without compromising the overall outer dimensions of the capsule device.

Compared to latch mechanisms of the prior art, the proposed latch mechanism offers improvements to the load bearing surfaces, and the risk of creep in the latch mechanism components is reduced.

Furthermore, the solution enables more effective wetting of the dissolvable latch support resulting in improved precision for timely releasing of the latching engagement.

In some embodiments, when in the pre-actuation configuration, the dissolvable latch support is primarily acted upon by compression forces exerted onto the dissolvable latch support by the latch arms being urged radially towards the dissolvable latch support.

In some embodiments, in the pre-actuation configuration, at least one pair of a latch arm and a retainer portion is structured to maintain, i.e. releasably retain, the actuation member.

In some forms of the capsule device, when assuming the pre-actuation configuration, the spring exerts a load on the actuation member, and the at least one pair of a latch arm and a retainer portion retains the actuation member relative to the base member against the load exerted by the spring.

In other forms, when the capsule device is taken into use, the spring will not initially exert a load on the actuation member, but may be operated, such as by a user-initiated step, to provide said load.

In some embodiments the plurality of latch arms is provided as two radially opposed latch arms arranged in a v-shaped configuration, and wherein the dissolvable latch support member is generally wedge shaped or shaped as a cone, or comprising a portion being shaped as a wedge or cone. In other embodiments, the number of latch arms may be three, four, five or more, wherein the latch arms are distributed around the axis.

In some embodiments, the plurality of retainer portions each comprise an inclined surface with a surface normal pointing proximally and radially inwards, and wherein the radially outwards facing latch surface of each latch arm comprises a correspondingly inclined surface.

The capsule housing defines an exterior surface. The inclined surfaces of the retainer portions may be formed to intersect with the capsule housing exterior surface.

The engagement interfaces between the radially outwards facing latch surfaces and the respective retainer portions may in certain embodiments be provided either as planar interfaces or single curvature interfaces, such as conically shaped interfaces.

In further embodiments, the engagement interfaces between the radially inwards facing latch surfaces and the respective radially outwards facing surfaces of the dissolvable latch support may be provided as planar interfaces or single curvature interfaces, such as conically shaped interfaces.

The exterior of the capsule housing defines a housing extreme proximal end surface. In some embodiments, the dissolvable latch support defines a dissolvable latch support proximal end surface, and wherein, in the pre-actuation configuration, the dissolvable latch support proximal end surface is located proximally relative to the housing exterior extreme proximal end. In alternative embodiments the dissolvable latch support proximal end surface is located distally within 2 mm, such as within 1.5 mm such as within 1.0 mm, such as within 0.5 mm from the housing exterior extreme proximal end.

In some embodiments, a fluid ingress opening is provided that enables ingress of a biological fluid through the capsule housing to the actuation compartment (B). In some embodiments, the fluid ingress opening is provided in the capsule housing proximal end, such as being arranged centrally on-axis, wherein the dissolvable latch support is disposed in the fluid ingress opening, e.g. to enable wetting of the dissolvable latch support.

Also, in some embodiments, one or more vents are provided which enables fluid and/or gas communication between the external environment of the self-righting capsule and the dissolvable latch support. In such embodiments, the vents may be formed so as to create gas or fluid to pass through windings of the helical spring. By providing two or more openings in the actuation compartment (B), the risk of gas, such as air, becoming trapped next to a surface of the dissolvable latch support will be decreased.

In some further forms, the distal portion of the actuation member comprises a cup-shaped geometry having a proximally facing cavity forming a spring seat for the distal end portion of the helical spring, wherein an exterior flange portion of the cup shaped geometry forms a proximal facing annular surface portion arranged in engagement with the annular seal when the actuation member is in the proximal position.

In some embodiments, the exit port is configured to maintain the sealing compartment (A) fluidically isolated from an external environment of the self-righting capsule until actuation of the self-actuating actuator.

The self-righting capsule may be provided in a form wherein a dose member is accommodated in the sealing compartment (A), the dose member being shaped as a solid therapeutic dosage form formed partly or entirely from a preparation comprising a therapeutic payload, the preparation being made from a dissolvable material that dissolves when inserted into the into tissue of the lumen wall to at least partly release the therapeutic payload into the blood stream.

In exemplary embodiments, the capsule device is configured for ingestion and for travelling into a lumen of a patient, the lumen having a lumen wall.

In such embodiments, the capsule device may be configured as a self-righting capsule, wherein when the self-righting capsule is at least partially supported by the tissue of the lumen wall, the self-righting capsule self-orients relative to gravity with the axis arranged vertically and the distal end pointing downwards.

In some forms the dosage form is attached to the actuation member, such as in the pre-actuation configuration. In other forms, the actuation member engages the dosage form during the stroke of the actuation member from the first position to the second position.

In some forms the dose member defines a tissue penetrating member.

In some forms the capsule device comprises a tissue penetrating member coupled to the actuation member, wherein the tissue penetrating member is disposed within the capsule housing when the actuation member assumes the first position and wherein the tissue penetrating member is advanced from the capsule housing and into the wall of the lumen as the actuation member moves from the first position to the second position.

In some forms the capsule device defines an ingestible device suitable for swallowing by a patient and travelling into a lumen of a gastrointestinal tract of a patient, such as the stomach or the small intestines. The capsule hosing of the device may be shaped and sized to allow it to be swallowed by a subject, such as a human. By the above arrangements an orally administered drug can be delivered safely and reliably into the stomach or intestinal wall of a living mammal subject, such as a human.

In some embodiments, the actuation mechanism is operable from a pre-actuation configuration, through an actuating configuration and end in an actuated configuration. The actuating configuration may be referred to as an intermediary configuration wherein the actuation mechanism releases the actuation member for movement towards the actuated configuration.

In further exemplary embodiments the self-righting capsule defines a capsule device comprising a capsule housing having an outside shape formed as a rounded object and defining an exterior surface. The capsule device further comprises said dose member having a needle or dart-shaped form and being formed partly or entirely from a preparation comprising a therapeutic payload, wherein the preparation is made from a dissolvable material that dissolves when inserted into tissue of the lumen wall. The actuation member may take the form as a hub that comprises an interface portion, wherein the dose member is held or attached relative to the interface portion of the hub. The capsule device is configured as a self-righting capsule having a geometric center and a center of mass offset from the geometric center along the axis, wherein when the capsule device is supported by the tissue of the lumen wall while being oriented so that the centre of mass is offset laterally from the geometric center the capsule device experiences an externally applied torque due to gravity acting to orient the capsule device with the axis oriented along the direction of gravity to enable the dose member to interact with the lumen wall at the target location. Upon entering into the actuating configuration, the hub moves along the axis thereby inserting the dose member into tissue. Subsequent to insertion, the dose member may at least partially dissolve and release one or more therapeutic agent(s) into the tissue.

In such embodiments the hub may be actuated to move the hub from a first position to a second position, and wherein the dose member is configured for detachment relative to the interface portion of the hub when the hub assumes the second position.

Non-limiting examples of a self-righting capsule device may include devices configured for actuation when the device is located in the stomach lumen of a patient. Other non-limiting examples may include devices configured for actuation when the device is located in the small intestines or the large intestines.

As used herein, the terms “drug” “therapeutic agent”, “dosage form”, “payload” or “therapeutic payload” is meant to encompass any drug formulation capable of being delivered into or onto the specified target site. The drug may be a single drug compound or a premixed or co-formulated multiple drug compound. Representative non-limiting drugs include pharmaceuticals such as peptides (e.g. insulins, insulin containing drugs, GLP-1 containing drugs as well as derivatives thereof), proteins, and hormones, biologically derived or active agents, hormonal and gene based agents, nutritional formulas and other substances in both solid, powder or liquid form. Specifically, the drug may be an insulin or a GLP-1 containing drug, this including analogues thereof as well as combinations with one or more other drugs.

### BRIEF DESCRIPTION OF THE DRAWINGS

In the following embodiments of the invention will be described with reference to the drawings, wherein:

FIG. 1 shows a perspective view of a first embodiment capsule device 100 in accordance with the invention, the device assuming a pre-actuation configuration,

FIG. 2 is a perspective partly cut view of the first embodiment capsule device 100 in the pre-actuation configuration, and

FIG. 3 shows a cross-sectional front view of a second embodiment capsule device 100 in accordance with the invention, the device assuming a pre-actuation configuration.

In the figures representing views of the different embodiments like structures are mainly identified by like reference numerals.

### DESCRIPTION OF EXEMPLARY EMBODIMENTS

When in the following terms such as “upper” and “lower”, “right” and “left”, “horizontal” and “vertical” or similar relative expressions are used, these only refer to the appended figures and not necessarily to an actual situation of use. The shown figures are schematic representations for which reason the configuration of the different structures as well as their relative dimensions are intended to serve illustrative purposes only. When the term member or element is used for a given component it generally indicates that in the described embodiment the component is a unitary component, however, the same member or element may alternatively comprise a number of sub-components just as two or more of the described components could be provided as unitary components, e.g. manufactured as a single injection moulded part. The terms “assembly” and “subassembly” do not imply that the described components necessarily can be assembled to provide a unitary or functional assembly or subassembly during a given assembly procedure but is merely used to describe components grouped together as being functionally more closely related.

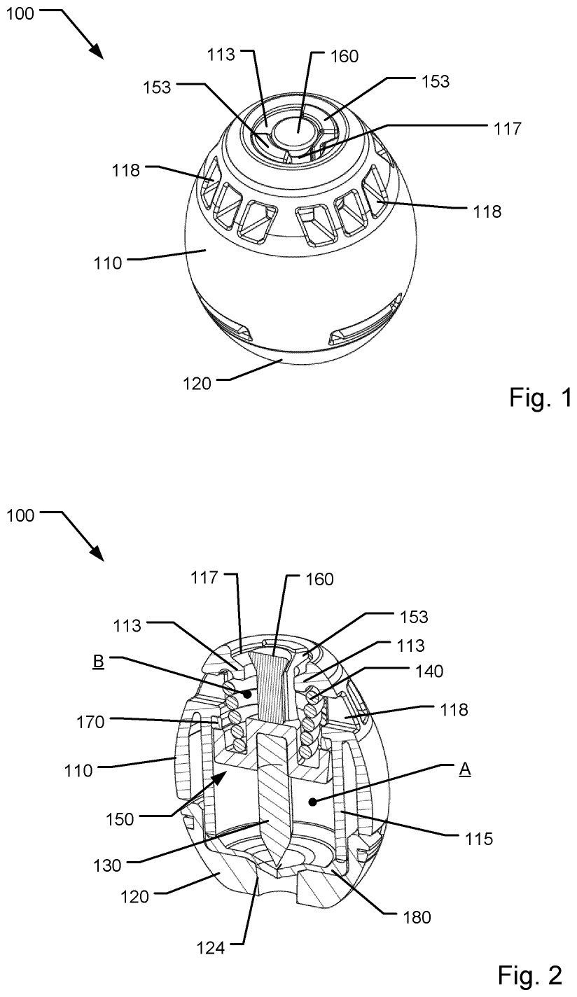

With reference to FIGS. 1, 2 and 3, first and second embodiments of a drug delivery device in accordance with different aspects of the invention will be described, the embodiments being designed to provide a capsule device 100 having a desired actuation configuration for deployment of a solid dosage form from an ingestible self-righting capsule device. The disclosed embodiments relates to a capsule device 100 suitable for being ingested by a patient to allow the capsule device to enter the stomach lumen, subsequently to orient relative to the stomach wall, and finally to deploy a solid dose payload for insertion at a target location in tissue of the stomach wall. Generally, at least a portion of the solid dose payload penetrates the tissue of the subject and at least a portion of a therapeutic agent within the payload dissolves into the tissue for uptake in the blood stream of the subject. For the capsule device 100 the general principle for orienting the capsule relative to the stomach wall may utilize any of the principles disclosed in WO 2018/213600 A1. The state shown in FIGS. 1, 2 and 3 all represents a pre-actuation configuration prior to swallowing of the capsule.

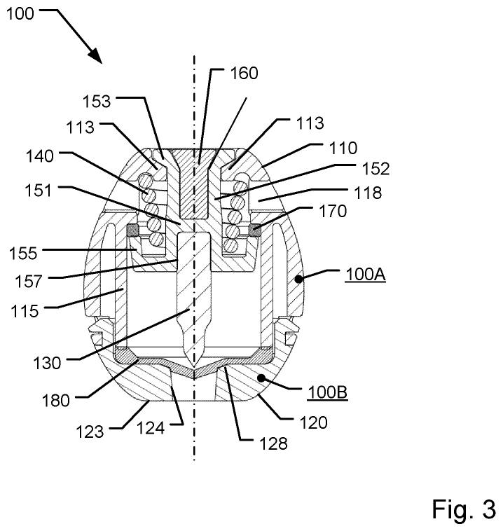

The first embodiment shown in FIGS. 1 and 2 provides for a capsule device wherein a dosage form accommodated within the device is shaped with a generally cylindrical main portion and with a distal portion formed with a pointed distal tip end providing improved penetration of the dosage form when pushed into tissue at a target location. The design of the second embodiment shown in FIG. 3 generally corresponds to the design of the first embodiment, however with the dosage form being shaped with a slightly more complex outer shape providing a contoured outline along the most distal part of the dosage form.

The ingestible self-righting capsule device 100 comprises a first portion 100A having an average density, a second portion 100B having an average density different from the average density of the first portion 100A (cf. FIG. 3). The capsule device 100 accommodates a tissue penetrating member forming a dosage form 130 for carrying an agent for release internally of a subject user that ingests the article. In the shown embodiment, the average density of capsule device prior to deployment is larger than that of gastrointestinal fluid, enabling the capsule device to sink to the bottom of the stomach lumen. The outer shape of the self-righting article is a gomboc shape, i.e. a gomboc-type shape that, when placed on a surface in any orientation other than a single stable orientation of the shape, then the shape will tend to reorient to its single stable orientation.

The capsule device shown includes an upper (proximal) capsule housing 110 which mates and attaches to a lower (distal) capsule housing 120. The upper capsule housing 110 and the lower capsule housing 120 together forms the capsule housing of the device. In the shown embodiment upper capsule housing 110 and lower capsule housing 120 are mounted relative to each other by way of a snap engagement. The capsule housing parts 110/120 define a shell having an interior hollow which accommodates the dosage form 130 and an actuation and propulsion mechanism, i.e. a self-actuating actuator. The latter comprises an energy source in the form of a pre-strained helical drive spring 140, and an actuation member in the form of hub 150 which holds and drives forward the dosage form 130 for payload delivery upon release of energy from the drive spring 140. The dosage form 130 is oriented along an actuation axis and configured for movement along the actuation axis. In the shown embodiment, the upper and lower capsule housing parts 110, 120 form generally rotation symmetric parts with the axis of symmetry arranged along the actuation axis. In the drawings, the device is oriented with the actuation axis pointing vertically, and with the dosage form 130 pointing vertically downwards towards an exit hole 124 arranged centrally in the lower capsule housing 120, the exit hole allowing the dosage form 130 to be transported through exit hole and moved outside the capsule device 100. The lower capsule housing part 120 includes a tissue engaging surface 123 which is formed as a substantially flat lower outer surface surrounding the exit hole 124.

Regarding suitable materials for the capsule housing for the embodiments shown, the upper capsule housing 110 may suitably be made from a low-density material, such as polycaprolactone (PCL), whereas the lower capsule housing 120 may be suitably made from a high-density material, such as 316L stainless steel. In other embodiments, other materials may be used.

In the shown embodiment, due to the density distribution of the entire capsule device 100, and due to the outside shape of the device, the capsule device 100 will tend to orient itself with the actuation axis substantially perpendicular to the surface (e.g., a surface substantially orthogonal to the force of gravity, a surface of a tissue such as the wall of the gastrointestinal tract). Hence, the capsule device seeks to orient relative to the direction of gravity so that the tissue engaging surface 123 faces vertically downward.

The interior of the upper capsule housing part 110 includes a guiding structure provided in form of an inner sleeve 115 which extends concentrically with the actuation axis from the upper part of the upper capsule housing 110 towards a proximally facing bottom surface 128 formed in the lower capsule housing part 120. The inner sleeve guiding structure serves for guiding the hub 150 during deployment. Furthermore, the inner sleeve 115 forms part of a sealing compartment (A) for dosage form 130.

Further, in the shown embodiments, an upper hollow portion of housing part 110 forms an actuation compartment (B) for accommodating portions of the self-actuating actuator. At the proximal end portion of housing part 110 a fluid ingress opening 117 is formed centrally (on-axis). Prior to actuation, the sealing compartment (A) is fluidically isolated from the actuation compartment (B).

A hub retaining structure 113 is provided as an inwardly extending round-going flange that is arranged concentrically with the actuation axis and which extends radially inwards relative to the fluid ingress opening 117 from the upper capsule housing part 110 and downwards along the actuation axis. The hub retaining structure 113 serves as a retaining geometry for releasably retaining the hub 150 against the drive force emanating from the pre-strained drive spring 140 arranged within the capsule. Referring mainly to FIG. 2, in the shown embodiment, the hub retainer structure 113 provides a conical retainer surface wherein a surface normal to the conical retainer surface points proximally and radially inwards. In the shown embodiment the conical retainer surface interfaces directly with the exterior rounded surface at a proximal end portion of upper capsule housing 110. In the shown first embodiment of capsule device 100, the conical retainer surface of hub retaining structure 113 forms an angle of approximately 60 deg. relative to the actuation axis.

The different geometric structures provided in the fluid ingress opening 117 are dimensioned so that the hub 150 is movable axially distally from the central opening when the hub assumes a released state but wherein the hub 150 cannot move axially distally relative to the central opening when the hub 150 assumes a state corresponding to the pre-actuation configuration. The structure defining the fluid ingress opening 117 allows gastric fluid to enter into contact with a fluid operated actuation mechanism. Furthermore, in the shown embodiments, a plurality of vents 118 are formed in the capsule housing part 110 to provide fluid ingress to and allow trapped air to escape from the fluid operated actuation mechanism. In the shown embodiments, twelve vents 118 are distributed around the axis. However, in other embodiments different numbers of vents may be provided, or even omitted.

In the shown embodiments, dosage form 130 defines a solid delivery member formed entirely or partly from a preparation comprising the therapeutic payload. In the shown embodiment, the solid delivery member is formed as a thin cylindrical rod which is shaped to penetrate tissue of the lumen wall, the cylindrical rod having a tissue penetrating end, i.e. a tip portion, and a trailing end opposite the tissue penetrating end. In the shown embodiment, the tissue penetrating end of the rod is pointed to facilitate easy insertion into mucosal tissue of the lumen wall whereas the trailing end, in the shown embodiment shown in FIG. 2, defines a truncated cylinder cut off by a 90-degree cut. A non-limiting example of a drug suitable for delivery by capsule device 100 is dried compressed API, such as insulin.

Referring mainly to FIG. 3, the hub 150 comprises an upper retaining part 151 configured for releasably retaining the hub relative to the capsule housing and a lower interface part 155 configured for holding the trailing end of the dosage form 130 in place. In the shown embodiment, the lower interface part 155 includes a downward open bore 157 that receives the trailing end of the dosage form 130 in a way so that the dosage form 130 is firmly attached within the bore. The lower interface part 155 further defines an annular spring seat. The spring seat is formed in a cup-shaped distal part of the hub, where the spring seat is encircled by a round-going flange 155 extending in the proximal direction from distal most end surface of the hub 150.

The upper retaining part 151 of the hub 150 forms a base portion which at a proximal end connects with two latches provided in the form of two independently deflectable latch arms 152. In the state shown in FIGS. 2 and 3, the latch arms 152 are symmetrically arranged around the actuation axis. Each latch arm 152 extends from the retaining part 151 proximally generally in parallel with the actuation axis and ends with an end portion 153 having an angle with respect to the axis so that the end portion extend inclined proximally and radially outwards from the base portion. The proximal end portions 153 of the two latch arms 152 are thus configured in a v-shaped configuration. Each latch arm 152 is resiliently movable in the radial inwards direction by a swivelling movement relative to the upper retaining part 151. The latch arms 152 each defines radially outwards facing latch surfaces configured to engage with respective portions of the conical retainer 113 in a latching engagement. Each of the latch arms 152 further includes a radially inwards facing latch surfaces configured for cooperating with a centrally disposed dissolvable latch support 160. In the embodiment shown the inclined engaging surfaces are formed as conical surfaces.

In the shown embodiment, the hub 150, i.e. the actuation member, is configured displaceable within the capsule housing from a proximal position to a distal position.

In the shown first embodiment, in the pre-actuation configuration shown in FIG. 1a, the angle of inclination of the radially outwards facing latch surface forms an angle of 60 deg. relative to the axis whereas the angle of inclination of the radially inwards facing latch surface forms an angle of 28 deg. relative to the axis. Hence, from the base portion 151 towards the free end of the latch arms, the lateral thickness of the end portions of each latch arm increases gradually towards a larger lateral thickness at the free end. In other embodiments, different angles of inclination may be used for the inclined surfaces. Also, the angle of inclination may be the same for the radially outwards facing and radially inwards facing surfaces or the difference in angles of inclination between the surfaces may be even larger.

In the shown embodiment of hub 150, the latch arms 152 connect to the base portion of upper retaining part 151 by means of a hinge section allowing the two latch arms, relative to the positions they assume in FIG. 3, to become deflected radially inwards towards a collapsed position wherein the latch arms either touch each other or are positioned with a minor radial spacing between them. Hence, upon deflecting radially inwards, the deflectable latch arms are allowed to release from the conical retainer surface of hub retaining structure 113 thereby allowing the proximal part of hub 150 to move distally relative to the capsule housing 110/120 through the central opening 117.

It is to be noted that FIG. 3 shows the hub 150 in the state corresponding to the pre-actuation configuration. However, prior to assembly of the capsule device 100, i.e. prior to insertion of hub 150 into upper capsule housing 110, the latch arms may assume a different orientation when they assume a relaxed state. Hence, during manufacturing of the hub 150, which may be manufactured by injection molding, such as by molding using a high-strength thermoplastic material such as polyoxymethylene (POM) or similar material, the latch arms 152 may be formed so that, when the latch arms are in a relaxed state the latch arms assume the collapsed position wherein the arms are collapsed radially towards each other. This enables easy assembling of the capsule device, by allowing the hub 150 to be inserted in the proximal direction through the central opening formed in the conical retainer surface of hub retaining structure 113 without requiring a separate step of moving the latch arms towards the collapsed state.

In the shown embodiments, the dissolvable latch support 160 is formed with a generally cylindrical distal portion and a generally cone-shaped proximal portion sized to be inserted between the two latch arms 152 in a wedging manner forcing the latch arms 152 in intimate contact with the conical retainer surface of the hub retainer structure 113. In the shown embodiment, the conical surface of the dissolvable latch support generally matches the radially inwards facing surface of the end portions of the of the latch arms with an angle of inclination of approximately 28 degrees relative to the central axis.

For the dissolvable latch support 160, different forms and compositions may be used. Non-limiting examples include pellets made from Sorbitol or Microcrystalline cellulose (MCC). Other non-limiting examples include injection moulded Isomalt pellets, compressed granulate Isomalt pellets, compressed pellets made from a granulate composition of Citrate/NaHCO3, or compressed pellets made from a granulate composition of Isomalt/Citrate/NaHCO3.

Such dissolvable latch support will become disintegrated when subjected to a liquid such as gastric juice. By carefully selecting the composition, the geometry of the dissolvable latch support and optionally exposure channels to ensure wetting of the dissolvable latch support, the release time can be controlled to occur within a chosen time delay after swallowing of the capsule device 100.

The first embodiment capsule device 100 additionally comprises a pair of sealing elements 170, 180 for maintaining the tissue interfacing component, i.e. the dosage form 130, fluidically isolated from the environment external to capsule device 100 prior to actuation. In the shown embodiment, the sealing compartment (A) is formed by inner sleeve 115, an upper sealing element 170 provided between the flange 155 of the hub 150 and an inwardly extending flange of capsule housing part 110, lower sealing element 180 and the proximal portion of hub 150.

The upper sealing element 170 is formed as a ring of soft pliable material, such an elastomeric material. In the shown embodiments, the upper sealing element 170 has been sized and shaped so as to be arranged radially outside the drive spring 140, thus encircling a portion of the spring 140. In the shown embodiment, the annular seal 170 is arranged with a radially outwards facing surface interfacing with a radially inwards surface of the guide sleeve. A proximally facing surface of sealing element 170 interfaces with a distally facing rim surface of the inwardly extending flange of capsule housing part 110. In the shown embodiment, upper sealing element 170 provides an annular seal disposed fixedly relative to the axially extending enclosure formed by inner sleeve 115 and maintains its position as the hub 150 moves from the proximal position to the distal position.

The further sealing element, i.e. the lower sealing element 180, forms part of an exit port for dosage form 130. The lower sealing element is provided as a fluidic gate configured to maintain the exit hole 124 fluidically blocked prior to actuation. In the shown embodiment, the sealing element 180 comprises an elastomeric seal member having a generally disc shaped form. An outer periphery of the sealing element 180 is mounted below the inner sleeve 115 and clamped above an annular proximally facing surface of lower capsule housing 120. As disclosed in US 2020/0129441 A1 the central area of the sealing element 180 may comprise a fluidic gate formed to provide a self-sealing valve, such as formed by one or more thin cuts (e.g., one or more thin slits) that extend partially or completely through a centrally located material portion of the fluidic gate.

The sealing elements 170 and 180 thus cooperate to form a compartment internally in capsule device 100 that serves, prior to actuation, to maintain the dosage form 130 fluidically isolated from biological fluid externally to capsule device 100 but allows the dosage form to penetrate easily through sealing element 180 at the time of actuation for payload delivery into tissue.

During assembly, after the latch arms of the hub 150 has been inserted fully proximally through the upper part of the central opening 117 formed in the conical retainer surface with the sealing element 170 axially compressed between the hub 150 and the upper housing part 110. The latch support 160 is forced axially in the distal direction in between the latch arms 152. Due to the conical interface between the dissolvable latch support 160 and the latch arms 152, the dissolvable latch support 160 is allowed to be moved distally in a wedging action while the latch arms 152 become spread radially outwards into engagement with the conical retainer surface 113a. At the end of this assembly step, the wedging action provides stiction between the dissolvable latch support 160 and the latch arms 152 resulting in mounting engagement where the dissolvable latch support 160 remains fixedly attached to the latch arms 152. This enables safe storage and handling without the risk of dissolvable latch support 160 becoming accidentally dismounted from the latch arms 152. As seen in FIG. 1, the top surface 162 of dissolvable latch support is located substantially flush with the proximal opening 117 defined by conical retainer surface. This serves to ensure adequate wetting of the dissolvable latch support 160 when submerged into gastric juice. In other embodiments, the top surface 162 may either be located proximally or located distally to the extreme proximal end of the upper capsule housing 110.

With regard to the above-mentioned drive spring 140, the drive spring 140 has been provided in form of a compression spring which stores energy so as to expand during the delivery stroke. The drive spring 140 is provided as a helically coiled conical spring arranged partially axially within the inner sleeve 115. The drive spring forms a wide proximal first end arranged to be seated against an upper spring seat formed in the most proximal portion of upper capsule housing part 110. This upper spring seat is formed radially within a diameter less than the interior diameter of the inner sleeve 115. A distal narrow second end of spring 140 is seated in the spring seat formed by the lower interface part 155 of the hub 150.

As part of assembling the capsule device 100 the drive spring 140 has been energized by axially compressing the drive spring 140 between the two spring seats. Hence, prior to actuation, the hub 150 is maintained under load from drive spring 140.

Turning now to the operation of the capsule device 100, reference will primarily be made to FIG. 3 which shows the initial state which represents the state the capsule device assumes during storage or just after ingestion. In this state, the actuator 150 assumes the pre-actuation configuration where the two latch arms 152 are maintained in the shown position by engagement with radially outwards facing surfaces of the dissolvable latch support 160 engaging the radially inwards facing latch surface of the latch arms. As a result, the radially outwards facing latch surface of the latch arms are kept in engagement with the conical retainer surface preventing the latch arms from sliding relative to the conical retainer surface. As the latch arms 152 are trapped proximally to the conical retainer surface the hub 150 cannot be moved distally even though the drive spring 140 exerts its full compression load onto the actuator 150.

The upper sealing element 170 engages the flange 155 as well as an inwardly extending flange of capsule housing part 110 to keep this interface fluid tight. Also, the lower sealing element 180 keeps the exit hole 124 fluid tight.

After ingestion of capsule device 100, the capsule device quickly sinks to the bottom of the stomach. Upon being supported by the stomach wall, due to the self-righting ability of the capsule device, the capsule device will quickly reorient to have its tissue interfacing surface 123 engaging the tissue stomach wall with the firing axis of the capsule device oriented virtually vertical, i.e. with the dosage form 130 pointing downwards. Dissolution of dissolvable latch support 160 has begun due to exposure to gastric fluid through fluid ingress opening 117 and/or vents 118. The support from dissolvable latch support 160 against the latch arms 152 will cease at a specific time after swallowing. The load of the drive spring 140 will cause the latch arms 152 to be gradually deflected radially inwards thereby allowing the latch arms to slide off from engagement with the conical retainer surface. At some point in time the latch arms 152 will reach their collapsed position where after the hub 150 with the dosage form 130 will become released from the hub retainer structure 113. This state corresponds to the actuating configuration. As drive spring 140 exerts a distally directed force onto hub 150, the hub and the dosage form 130 are caused to travel unhindered towards the exit hole 124 with the dosage form penetrating the lower sealing element 180 and further into mucosal tissue at the target location. In the actuated configuration, a proximally facing hub stop surface 128 arranged at the bottom part of capsule housing 120 prevents the hub 150 from moving further distally.

In situation of intended use, the dosage form 130 is inserted into tissue of the lumen wall where it will anchor generally in a direction along the actuation axis. As discussed above, depending on the specific design of the capsule device, the dosage form 130 may be released actively from the remaining parts of the capsule at the end of the insertion stroke. When the capsule device 100 has delivered the intended dose the capsule will release relative to the deposited dosage form 130 which remains inside the tissue wall for release of therapeutic agent into the blood stream of the subject.

Although not shown in the embodiments disclosed herein, any of the embodiments may be modified to include a mechanism for separating the dosage form 130 from the hub 150 upon the assembly of the dosage form 130 and the hub 150 arriving at the most distal position in the capsule housing. Suitable non-limited principles may include the principles disclosed in WO 2020/157324 A1 wherein a ram (similar to a hub) becomes tilted at the end of the insertion stroke for detaching the tissue inserted portion of the delivery member from the ram.

Alternatively, the capsule may be held stationary for a prolonged time allowing the dosage form 130 to release a therapeutic agent into the blood stream of the subject as the capsule is held stationary relative to the tissue. In any of these cases, subsequently to drug delivery, the remaining parts of the capsule device will travel out through the digestive system of the user and be disposed of.

Due to the design of the actuation mechanism including a partly conically shaped dissolvable latch support and the v-shaped configuration of the latch arms in combination with the shape of the drive spring which includes a tapering outside diameter with a large diameter first end, the actuation mechanism and the drive spring has been designed to axially overlap each other. The said combination of features allows the load bearing surfaces of the actuator mechanism to be optimized, i.e. the interfaces between the hub retainer structure 113 and the latch arms 152 and the interfaces between the latch arms 152 and the dissolvable latch support 160. Compared to previously suggested designs disclosed in the art, the spring force of the drive spring has been increased without compromising the load bearing surfaces, and at the same time reducing the risk of creep in the parts forming the actuator mechanism. Hitherto, corresponding benefits were only available by compromises being made in the exterior size of the capsule.

It is to be noted that further not shown embodiments in accordance with the invention may include actuator mechanisms having actuator interfaces formed differently than the conical shaped interface surfaces shown in connection with the first and, second embodiments. For example, the actuator interfaces may be formed with planar surfaces instead of conical surfaces, either at the interface between the hub retainer structure 113 and the latch arm 152 and/or between the latch arm 152 and the dissolvable latch support 160. For such embodiment having two radially opposed latch arms, the dissolvable latch support 160 may be formed as partly forming a wedge having two planar surfaces intersecting each other at the sharp edge of the wedge.

Furthermore, the number of latch arms may be different than two, such as three, four or even more individual latch arms. In certain embodiments, the plurality of latch arms may be disposed equally around the actuation axis, although this may not be strictly necessary for any embodiment in accordance with the principles of the present invention.

The above described variant of interfaces between the dosage form 130 and the hub 150 are only exemplary and other configurations may be used instead. The detachable attachment between the dosage form and the hub may be obtained by using a friction or press fit. Alternatively, an adhesive may be used at the interface, such as sucrose. Still alternatively, the attachment may be obtained by initially wetting the dosage form and utilizing inherent stiction between the hub and the dosage form. In situation of use, upon the hub reaching its final destination, detachment may occur at the interface between the dosage form and the hub. In other embodiments, a desired detachment may be obtained by detaching a major portion of the dosage form from the remaining dosage form being still adhered or fastened to the hub. In some embodiments, the dosage form includes a weakened point which determines the point of separation. In still further embodiments, the hub and the dosage form may be formed as a unitary component all made of a composition containing API, and wherein the intended dosage form to be pushed out from capsule device is separated from the hub portion. Also, in alternative embodiments, the payload may act as a hub by itself to be fully transported away from the capsule device.

Although the above description of exemplary embodiments mainly concern ingestible capsules for delivery in the stomach, the present actuation principle generally finds utility in capsule devices for lumen insertion in general, wherein a capsule device is positioned into a body lumen, and wherein a fluid activates an actuation mechanism by dissolving a dissolvable latch support for bringing a component from a first configuration into a second configuration, such as from a first position into a second position. Non-limiting examples of capsule devices may include capsule devices for intestinal delivery of a payload or drug either by delivery into the intestinal lumen or into the tissue wall of an intestinal lumen.

In the above description of exemplary embodiments, the different structures and means providing the described functionality for the different components have been described to a degree to which the concept of the present invention will be apparent to the skilled reader. The detailed construction and specification for the different components are considered the object of a normal design procedure performed by the skilled person along the lines set out in the present specification.

1. A self-righting capsule suitable for ingestion into a lumen of a patient, the lumen having a lumen wall, the self-righting capsule comprising:
a capsule housing defining a central axis and having proximal and distal ends,
a sealing compartment within the capsule housing for accommodating a dose member shaped as a solid therapeutic dosage form formed partly or entirely from a preparation comprising a therapeutic payload, wherein the sealing compartment comprises an axially extending enclosure having a distal end provided with an exit port and a proximal end, and
an actuation compartment fluidically isolated from the sealing compartment and at least partially accommodating a self-actuating actuator,
wherein the self-actuating actuator comprises:
an actuation member arranged for axial displacement relative to the axially extending enclosure from a proximal position to a distal position thereby cooperating with the dose member for moving the dose member distally from a non-exposed position within the sealing compartment to an exposed position wherein at least a portion of the dose member is exposed through the exit port,
a helical spring for displacing the actuation member, wherein the helical spring is strained to exert a load on the actuation member for displacement from the proximal position to the distal position,
a latch component coupled to the actuation member for releasably maintaining the actuation member latched in the proximal position, and
a dissolvable latch support disposed in the actuation compartment, the dissolvable latch support being at least partially dissolvable when subjected to a biological fluid entering the actuation compartment, wherein the dissolvable latch support, prior to dissolution, maintains latching of the actuation member in the proximal position by supporting the latch component, and wherein at least partial dissolution of the dissolvable latch support causes support of the latch component to cease thereby enabling the actuation member to be displaced towards the distal position,
wherein the self-righting capsule comprises a sealed interface between the actuation member and the axially extending enclosure that, with the actuation member assuming the proximal position, maintains the sealing compartment fluidically isolated from the actuation compartment, wherein the sealed interface comprises an annular seal arranged radially outwards relative to at least a portion of the helical spring.

2. The self-righting capsule as in claim 1, wherein the helical spring comprises a plurality of windings, wherein a distal portion of the windings defines an outside diameter of first magnitude, and wherein the annular seal comprises a radially inwards facing surface defining an inner diameter of second magnitude larger than the first magnitude.

3. The self-righting capsule as in claim 1, wherein the helical spring is provided as a coiled tapered spring having a wide proximal end coupled to the capsule housing and a narrow distal end coupled to actuation member.

4. The self-righting capsule as in claim 1, wherein the annular seal is arranged in axially overlapping relationship with a portion of the helical spring, at least when the actuation member assumes the proximal position, and/or at least when the actuation member assumes the distal position.

5. The self-righting capsule as in claim 1, wherein the annular seal is arranged with a radially outwards facing surface interfacing with a radially inwards surface of the axially extending enclosure.

6. The self-righting capsule as in claim 1, wherein the annular seal is disposed fixedly relative to the axially extending enclosure as the actuation member moves from the proximal position to the distal position.

7. The self-righting capsule as in claim 1, wherein the actuation member comprises a distal portion coupled to the dose member and wherein the latch component is attached to or formed integrally with the distal portion, the latch component protruding proximally from the distal portion of the actuation member.

8. The self-righting capsule as in claim 7, wherein the latch component comprises one or more latch arms attached to, or being integrally formed with, the distal portion of the actuation member, the one or more latch arms being formed to extend proximally from the distal portion of the actuation member to cooperate with corresponding retainer portions associated with the capsule housing.

9. The self-righting capsule as in claim 8, wherein at least one of said one or more latch arms is formed as a laterally movable latch arm defining a blocking portion that cooperates with a corresponding retainer portion in latching engagement, wherein, prior to dissolution of the dissolvable latch support, the latch arm is supported by the dissolvable latch support, which upon dissolution ceases to support the latch arm thereby enabling lateral movement of the latch arm to release the latching engagement.

10. The self-righting capsule as in claim 8, comprising a plurality of latch arms, wherein each latch arm defines radially outwards and radially inwards facing latch surfaces, and wherein each latch arm is radially deflectable,
wherein the dissolvable latch support is arranged centrally on-axis, wherein the retainer portions are arranged coaxially around the dissolvable latch support, and wherein the latch arms are arranged radially in between,
wherein, prior to dissolution of the dissolvable latch support, the radially outwards facing latch surfaces engage with respective retainer portions in a latching engagement, and the radially inwards facing latch surfaces are in supporting engagement with respective radially outwards facing surfaces of the dissolvable latch support thereby restricting the latch arms from moving radially inwards and preventing release of the latching engagement,
wherein, prior to dissolution, the latch arms extend proximally and radially outwards from the distal portion of the actuation member so that the radially inwards facing latch surface of each latch arm is inclined relative to the axis, and wherein the respective radially outwards facing surfaces of the dissolvable latch support are inclined thereby supporting the respective latch arm along the radially inwards facing latch surface, and
wherein, when the dissolvable latch support has become at least partially dissolved, the latch arms are allowed to move radially inwards thereby releasing the latching engagement and allowing the helical spring to move the actuation member towards the distal position.

11. The self-righting capsule as in claim 1, wherein a fluid ingress opening is provided in the capsule housing proximal end, such as being arranged centrally on-axis, wherein the dissolvable latch support is disposed in the fluid ingress opening.

12. The self-righting capsule as in claim 1, further comprising one or more vents to provide fluid and/or gas communication between the environment externally of the self-righting capsule and the dissolvable latch support.

13. The self-righting capsule as in claim 1, wherein the distal portion of the actuation member comprises a cup-shaped geometry having a proximally facing cavity forming a spring seat for the distal end portion of the helical spring, wherein an exterior flange portion of the cup shaped geometry forms a proximal facing annular surface portion arranged in engagement with the annular seal when the actuation member is in the proximal position.

14. The self-righting capsule as in claim 1, wherein the exit port is configured to maintain the sealing compartment fluidically isolated from an external environment of the self-righting capsule until actuation of the self-actuating actuator.

15. The self-righting capsule as in claim 1, and further comprising a dose member accommodated in the sealing compartment, the dose member being shaped as a solid therapeutic dosage form formed partly or entirely from a preparation comprising a therapeutic payload, the preparation being made from a dissolvable material that dissolves when inserted into the into tissue of the lumen wall to at least partly release the therapeutic payload into the blood stream.
